(19) United States
(12) Patent Application Publication
(10) Pub. No.: US 2011/0223216 A1
(43) Pub. Date: Sep. 15, 2011

(54) Nanoparticles and Porous Particles and Methods of Making the Same

(75) Inventors: Sandro R.P. Da Rocha, Troy, MI (US); Libo Wu, Detroit, MI (US)

(73) Assignee: WAYNE STATE UNIVERSITY, DETROIT, MI (US)

(21) Appl. No.: 13/129,804

(22) PCT Filed: Nov. 17, 2009
(86) PCT No.: PCT/US09/64863

§ 371 Date: May 17, 2011

Related U.S. Application Data
(60) Provisional application No. 61/199,519, filed on Nov. 17, 2008.
(60) Provisional application No. 61/199,518, filed on Nov. 17, 2008.

(51) Int. Cl. A61K 9/14; A61K 9/12; A61K 38/28; A61K 31/137; A61K 31/58; A61P 11/00; A61P 3/10; A61P 35/00; A61P 11/06; A61P 11/08; A61P 9/12; A61P 31/18; A61P 35/02; A61P 7/06; A61P 7/00; A61P 19/02; A61P 9/00; B82Y 5/00

(52) U.S. Cl. 424/400; 424/45; 514/5.9; 514/653; 514/174; 977/773; 977/906

(57) ABSTRACT

The subject matter disclosed herein relates to compositions and methods for engineering porous particles for aerosol formulations for pulmonary drug delivery. Certain embodiments disclosed herein further relate to methods for stabilizing suspension-based formulations in hydrofluoroalkane propellants with nanoparticles.

### STATEMENT OF GOVERNMENT INTEREST

This work was supported in part by National Science Foundation grant number CBET 0553537. The government has certain rights to the invention.

### FIELD

The subject matter disclosed herein relates to compositions and methods for engineering porous particles for aerosol formulations for pulmonary drug delivery. Certain embodiments disclosed herein further relate to methods for stabilizing suspension-based formulations in hydrofluoroalkane propellants with nanoparticles.

### BACKGROUND

Pressurized metered-dose inhalers (pMDIs) are relatively inexpensive and widely used in pulmonary drug delivery. (Bell, Newman, Expert Opin. Drug Delivery 4:215-234 (2007). The propellant is the major constituent of pMDIs, where the active ingredients are either solubilized (solution formulations) or dispersed (suspension formulations). Suspension-based formulations account for almost half of the commercially available pMDIs, and have certain advantages over solution-based formulations such as the possibility of delivering higher dosages, and improved chemical stability of the therapeutic ingredients. pMDIs may also contain non-active excipients such as cosolvents, amphiphiles and flavors.

Due to the ozone depleting potential of chlorofluorocarbon (CFC), hydrofluoroalkanes (HFAs) have been selected as environmentally acceptable propellant alternatives, even though HFAs are known greenhouse gases. (McDonald and Martin, Int. J. Pharm. 201:89-107 (2000)). While HFAs have some similarities to CFCs, the excipients used in FDA-approved CFC-based pMDI formulations are generally not compatible with HFAs due to the significant differences in physicochemical properties between these two classes of solvents. (Courrier et al., Crit. Rev. Ther. Drug Carrier Syst. 19:425-498 (2002)). Ethanol is, therefore, typically employed to enhance the solubility of excipients in HFAs. (Rogueda, Expert Opin. Drug Delivery 2:625-638 (2005)). However, the presence of co-solvents in the formulation may enhance the solubility of the active drug ingredients resulting in reduced chemical stability. (Tzou et al., J. Pharm. Sci. 86:1352-1357 (1997)). Cosolvents also decrease vapor pressure of the propellant mixture, thus affecting the aerosol performance of the corresponding formulations. (Vervaeit and Byron, Int. J. Pharm. 186:13-30 (1999)).

Many alternative formulations have been proposed to address the reformulation issues that have affected the transition to HFA-based pMDIs. In the case of PROVENTIL® HFA (Schering-Plough Corporation), as an example, the active ingredient (salbutamol base) was replaced by its salt, which has lower solubility in ethanol, an excipient used in that formulation. (Tzou et al., J. Pharm. Sci. 86:1352-1357 (1997)). Changing the chemistry of the drug ingredient, however, is not always feasible. One potential alternative that has been evaluated is the development novel HFA-philic excipients that have high solubility in propellant HFAs, and thus do not require the use of ethanol in the formulation. (Wu and da Rocha, “Biocompatible and Biodegradable Copolymer Stabilizers for Hydrofluoroalkane Dispersions: A Colloidal Probe Microscopy Investigation,” Langmuir 23:12104-10 (2007); Traini et al., Int. J. Pharm. 320: 58-63 (2006); Stefely et al., Respir. Drug Delivery VII: 83-90 (2000); Peguin et al., “The Ester Group: How Hydrofluroalkane-philic is it.” Langmuir 23:8291-8294 (2007)). Within that context, recent investigations addressing solvation in HFAs have been relevant. (Wu et al., J. Phys. Chem. B 111:8096-8104 (2007)). Combined microscopic computational and experimental approaches have been employed to quantify HFA-philicity for several chemistries of pharmaceutical relevance. The obtained solvation information has in turn been used to design novel amphiphiles capable of stabilizing dispersions in the low dielectric HFAs. Such knowledge has also been relevant in the development of novel particle engineering approaches that allow for the direct modification of the surface chemistry of the particles, resulting in enhanced physical stability in the propellant and improvement in the characteristics of the corresponding aerosol formulations. (Liao et al., Int J Pharm. 304:2939 (2005); Jones et al., J. Controlled Release 115:1-8 (2006); Dickinson et al., J. Drug Targeting 9:295-302 (2001); Dellamary et al., Pharm Res. 17:168-174 (2000); Wu and da Rocha, “Novel Propellant-driven Inhalation Formulations: Engineering Polar Drug Particles with Surface-trapped Hydrofluoroalkane-philes,” Eur J Pharm Sci 33:146-258 (2007)).

Traditionally, spray-drying has been the standard technique for preparing porous particles, but inadequate results have been obtained. Thus, there remains a need in the art to develop methodologies for engineering porous polar drug particles that result in pMDI formulations with enhanced aerosol characteristics while maintaining minimum amounts of non-active ingredients. Furthermore, there remains a need in the art to develop stabilized suspension-based formulations in HFA or other gas propellants with nanoparticles. Finally, there remains a need in the art for increased bioavailability, and low toxicity suspensions for delivery. Certain embodiments disclosed herein fulfill these needs, as well as others in the art.

### SUMMARY

Embodiments disclosed herein relate to processes for preparing an active agent porous particle, the process comprising contacting at least one active agent with an aqueous dispersion, emulsifying the active agent and aqueous dispersion, and diffusing the active agent and aqueous dispersion in an organic solvent under suitable conditions to form an active agent porous particle. In certain embodiments, the suitable conditions can be one or more of, without limitation: the presence of at least one surfactant, and the presence of at least one porosity agent. In certain other embodiments, the porosity agent can be one or more of without limitation: lecithin, glycolipids, phospholipids, and triglycerides.

Certain of these embodiments relate to processes wherein the surfactant is selected from one or more of, without limitation, ionic or non-ionic surfactants including without limitation: phospholipids, glycolipids, ganglioside GM1, sphingomuelin, phosphatidic acid, cardiolipin, lipids bearing polymer changes such as polyethylene glycol, chitin, hyaluronic acid, polyvinylpyrrolidone, lipids bearing sulfonated mono-, di-, and polysaccharides, fatty acids such as palmitic acid, stearic acid, oleic acid, cholesterol, cholesterol esters, sorbitan esters, polyoxyethelene, oleyl polyoxyethylene ether, glycerol esters, sucrose esters, lauryl polyoxyethylene ether, block copolymers and sodium (bis-2-ethylhexyl) sulfosuccinate AOT.

Certain other of these embodiments relate to processes wherein the active agent is one or more of, without limitation: antibiotics, antibodies, antiviral agents, anepileptic agents, analgesics, anti-inflammatory agents and bronchodilators, polysaccharides, steroids, hypnotics and sedatives, psychic energizers, tranquilizers, anticonvulsants, muscle relaxants, anti-Parkinson agents, analgesics, anti-inflammatory agents, muscle contractant agents, antimicrobial agents, anti-malarial agents, hormonal agents including contraceptives, sympathomimetics, amino acids, peptides, polypeptides, and proteins capable of eliciting physiological effects, diuretics, lipid regulating agents, anti-androgenic agents, anti-parasitic agents, neoplastic agents, anti-neoplastic agents, angiogenic agents, anti-angiogenic agents, hypoglycemic agents, nutritional agents and supplements, growth supplements, fats, anti-enteritis agents, electrolytes, vaccines, salbutamol sulfate, terbutaline hemisulfate, therapeutic biomolecules, formoterol, corticosteroids, fluticasone, chromolyn sodium, pain relievers insulin, calcitonin, erythropoietin, Factor VIII, Factor, ceredase, cerezyme, cyclosporine, granulocyte colony stimulating factor, alpha-1 proteinase inhibitor, elcatonin, granulocyte macrophage colony stimulating factor, growth hormone, human growth hormone, growth hormone releasing hormone, heparin, low molecular weight heparin, Interferon α, Interferon β, Interferon γ, Interleukin-2, luteinizing hormone releasing hormone, leuprolide, somatostatin, somatostatin analogs including octreotide, vasopressin analog, follicle stimulating hormone, immunoglobulins, insulin-like growth factor, insulintropin, interleukin-1 receptor antagonist, interleukin-3, interleukin-4, interleukin-6, macrophage colony stimulating factor, nerve growth factor, parathyroid hormone, thymosin α-1, Interleukins, interleukin receptors, soluble cytokines, soluble cytokine receptors, respiratory syncytial virus antibody, tetanus toxoid, lysozyme, other enzymes, deoxyribonuclease, bactericidal/permeability increasing protein, anti-CMV antibody, 13-cis retinoic acid, nicotine, nicotine bitartrate, gentamicin, ciprofloxacin, amphotericin, amikacin, tobramycin, pentamidine isethionate, albuterol sulfate, metaproterenol sulfate, beclomethasone dipropionate, triamcinolone acetamide, budesonide acetonide, ipratropium bromide, flunisolide, fluticasone, fluticasone propionate, salmeterol xinofoate, formeterol fumarate, cromolyn sodium, ergotamine tartrate, nucleic acids, peptides, polypeptides, proteins, amino acids nucleotides, DNA, RNA, tRNA, mRNA, rRNA, shRNA, microRNA, and pharmacologically acceptable salts thereof.

Still certain other embodiments relate to processes wherein the organic solvent is one or more of, without limitation: ethyl acetate, methanol, ethanol, 1-propanol, 2-propanol, acetonitrile, N,N′-dimethylformamide and tetrahydrofuran. Certain other embodiments relate to an active agent porous particle prepared by a process described herein.

Certain embodiments relate to processes for preparing an active agent porous particle, the process comprising contacting an active agent with an aqueous dispersion, emulsifying the active agent and aqueous dispersion in the presence of AOT, and diffusing the active agent and aqueous dispersion in an organic solvent comprising ethyl acetate under suitable conditions to form an active agent porous particle, wherein the active agent comprises a therapeutic drug. Certain other embodiments relate to methods for diagnosing or treating a therapeutic disease or condition comprising contacting the active agent porous particle prepared by a process described herein with a subject in need thereof, wherein the therapeutic disease or condition is one or more of, without limitation: chronic pulmonary diseases, lung cancer, cystic fibrosis, pulmonary fibrosis, asthma, bronchitis, pneumonia, pleurisy, emphysema, pulmonary fibrosis, diabetes, interstitial lung disease, sarcoidosis, chronic obstructive pulmonary disease, infant respiratory distress syndrome, adult respiratory distress syndrome, pulmonary actinomycosis, pulmonary alveolar proteinosis, pulmonary anthrax, pulmonary arteriovenous malformation, pulmonary edema, pulmonary embolus, pulmonary histiocytosis X (eosinophilic granuloma), pulmonary hypertension, pulmonary nocardiosis, pulmonary tuberculosis, pulmonary veno-occlusive disease, rheumatoid lung disease, hypertension, HIV-AIDS, leukemia, lymphoma, cancer, systemic vasculitis, anemia, stem cell transplants, hemophilia, polycythemia vera, thalassemia, thrombocytopenia, von Willebrand disease, arthritis, vascular diseases, heart conditions, and heart disease.

Still other embodiments relate to aerosolized pharmaceutical compositions comprising an active agent porous particle prepared by a process disclosed herein. Certain embodiments relate to processes for preparing a stabilized suspension-based aerosolized formulation, the processes comprising preparing nanoparticles, modifying the surface of the nanoparticles, and suspending the nanoparticles in an aerosolized formulation comprising at least one active agent, thereby preparing a stabilized suspension-based aerosolized formulation.

In certain embodiments, the diameter of the nanoparticles comprise without limitation 1 nm, 5 nm, 10 nm, 15 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 350 nm, 400 nm, 450 nm, or 500 nm in size.

In certain embodiments, the aerosolized formulations further comprise hydrofluoroalkane propellant. In still other embodiments, at least one active agent is one or more of, without limitation: insulin, budesonide, salbutamol sulfate, and terebutaline hemisulfate.

Certain embodiments disclosed herein relate to methods of diagnosing or treating a therapeutic condition or disease comprising administering a stabilized suspension-based aerosolized formulation prepared by processes disclosed herein, comprising contacting a subject in need thereof with the aerosolized formulation, wherein the therapeutic condition or disease is one or more of, without limitation: chronic pulmonary diseases, lung cancer, cystic fibrosis, pulmonary fibrosis, asthma, bronchitis, pneumonia, pleurisy, emphysema, pulmonary fibrosis, diabetes, interstitial lung disease, sarcoidosis, chronic obstructive pulmonary disease, infant respiratory distress syndrome, adult respiratory distress syndrome, pulmonary actinomycosis, pulmonary alveolar proteinosis, pulmonary anthrax, pulmonary arteriovenous malformation, pulmonary edema, pulmonary embolus, pulmonary histiocytosis X (eosinophilic granuloma), pulmonary hypertension, pulmonary nocardiosis, pulmonary tuberculosis, pulmonary veno-occlusive disease, rheumatoid lung disease, hypertension, HIV-AIDS, leukemia, lymphoma, cancer, systemic vasculitis, anemia, stem cell transplants, hemophilia, polycythemia vera, thalassemia, thrombocytopenia, von Willebrand disease, arthritis, vascular diseases, heart conditions, and heart disease.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 shows a scanning electron microscope image of chitosan-PLA nanoparticles, according to one exemplary embodiment disclosed herein;

FIG. 2 shows the dispersion stability of chitosan-PLA nanoparticles and insulin as an active agent (2 mg/ml) in HFA227 at 298K and saturation pressure of the propellant, where (A) is bare insulin, (B) is insulin in the presence of 1 mg/ml chitosan-PLA nanoparticles; and (C) is 2 mg/ml chitosan-PLA nanoparticles;

FIG. 3 shows the scanning electron microscope image of chitosan-PLA nanoparticles stabilized insulin HPFP formulation where the image was obtained following the evaporation of HPFP, and the nanoparticle concentration was mg/ml; inset top left indicates high magnification image of chitosan-PLA nanoparticle stabilized insulin, and inset top right indicates bare insulin particles;

FIG. 4 indicates the aerosol characteristics of Budesonide formulations as determined by ACI in vitro standard, where the use of nanoparticles as disclosed herein increased the fine particle fraction that is delivered to the deep lungs by 20% compared to the formulation containing solely micronized budesonide;

FIG. 5 is a schematic illustration of the preparation process of porous particles by emulsification diffusion;

FIG. 6 is a scanning electron microscopy image of salbutamol sulfate particles prepared with different surfactants at fixed lecithin (20 mg/ml) and surfactant (2.5 mg/ml) concentration (A) oleic acid, (B) L81, (C) AOT;

FIG. 7 shows the interfacial tension of water with ethyl acetate in the presence of oleic acid and AOT;

FIG. 8 is a scanning electron microscopy image of salbutamol sulfate particles prepared at fixed lecithin concentration (20 mg/ml) with AOT at the concentration of (A) 0 mg/ml, (B) 0.5 mg/ml; (D) 1.5 mg/ml; (E) 2.5 mg/ml, and salbutamol sulfate particles were prepared at fixed AOT concentration (0.5 mg/ml) with lecithin at the concentration of (B) 20 mg/ml, (C) 35 mg/ml; and at fixed AOT concentration (2.5 mg/ml) with lecithin at the concentration of (F) 5 mg/ml and (E) 20 mg/ml;

FIG. 9 shows the dispersion stability of salbutamol sulfate particles in HFA227 and HFA134a, where (a) solid salbutamol sulfate particles were prepared by emulsification-diffusion without using AOT and lecithin; and (b) porous salbutamol sulfate particles prepared with AOT concentration of 0.5 mg/ml and lecithin of 35 mg/ml;

FIG. 10 shows the aerodynamic particle size distribution of Ventolin® HFA (GlaxoSmithKline) solid salbutamol sulfate and porous salbutamol sulfate particle formulations prepared with AOT concentration of 0.5 mg/ml and lecithin of 35 mg/ml in HFA 134a (2 mg/ml) (a) without spacer; (b) with spacer. (AC refers to actuator, IP refers to induction port, SP refers to spacer, and F refers to filter).

### DETAILED DESCRIPTION

This disclosure is directed to nanoparticle engineering, particle-surface engineering (particularly porous particle engineering) and stabilization of suspension-based formulations in inhaled therapies, including, without limitation, gas propellants, and more particularly hydrofluoroalkane (HFA) gas.

Nanoparticles can be used as drug carriers due to their high stability, high carrier capacity, ability to incorporate both hydrophilic and hydrophobic substances, and feasibility of variable routes of administration, including oral application and inhalation. Nanoparticles can also be designed to allow controlled or sustained drug release from a matrix. Thus, nanoparticles allow for improved drug bioavailability and reduction in dosing frequency.

Particle-surface engineering approaches, particularly for porous particles, can have certain advantages when compared to surfactant-stabilized dispersions, but the requirement of extra excipients for the formulation still poses a problem because they are generally not FDA approved. Morphology design can overcome this shortcoming. As described herein, the primary effort of current morphology design is directed to developing porous particles. (Dellamary et al., Pharm. Res. 17:168 (2000); Edwards et al., J. Appl. Physiol. 85: 379-385 (1989); Edwards et al., Science 276: 1868-1871. (1997)) The porosity in the particles allows the propellant to penetrate into the particles, which gives rise to not only a close particle density with the propellant, but also a reduced Hamaker constant and corresponding minimized van der Waals attractive force. (Dellamary et al., Pharm. Res. 17:168-174 (2000)). However, the content of active ingredients in the porous particles obtained from spay-drying accounts for 50 wt % or less. (Dellamary et al., Pharm. Res. 17:168-174 (2000)).

Certain embodiments disclosed herein relate to methods for engineering porous drug particles with enhanced physical stability and aerosol characteristics in HFA or other gas propellants for use in pMDI formulations or other inhalation formulations. Certain embodiments relate to preparing drug particles (particularly of polar nature) containing excipients that can be later removed, thus generating a porous structure. Such methods allow for the propellant to penetrate the porous drug particle, thus enhancing the physical stability of the formulation. In certain embodiments, such formulations are otherwise unstable.

In other embodiments, the nanoparticles can be engineered to be porous particles. In certain embodiments, the non-active excipients remaining in the engineered particle can be limited to those in FDA-approved formulations.

A modified emulsification-diffusion technique was utilized to prepare porous particles. Salbutamol sulfate and terbutaline hemisulfate were used as the model polar drugs. The effect of preparation parameters on the morphology of the porous drug particles was also evaluated. The resulting physical (bulk) stability of the dispersions in 1,1,1,2-tetrafluoroethane (HFA134a) and 1,1,1,2,3,3,3-heptafluoropropane (HFA227), and the performance of the corresponding aerosols were examined, and directly compared to non-porous particles with the same morphology. The methods disclosed herein can improve wt % of active agents over standard particle preparation processes, and also in embodiments herein do not need the use of co-solvents.

It is further understood that both the nanoparticle formulations and the porous particle formulations may be encapsulated, combined with other formulations (whether nanoparticle formulations and/or porous particle formulations), attached to medical devices or other surfaces, or administered by various routes (including but not limited to inhalation, oral, topical, intravenous, intraperitoneal, intramuscular, vaginal, rectal, buccal, or other forms of administration).

In certain embodiments described herein, the nanoparticle and/or porous particle formulations can exhibit increased homogenous mixtures of the active agent formulation. In other embodiments, the nanoparticle and/or porous particle formulations can exhibit increased stability of the dispersion. In still other embodiments, the nanoparticle and/or porous particle formulations can exhibit increased efficiency or efficacy of delivery, and/or increased continuous or sustained delivery of the active agent(s).

### Nanoparticles

The term “nanoparticle” has been used to refer to nanometer-size devices consisting of a matrix of dense polymeric network (also known as nanospheres) and those formed by a thin polymeric envelope surrounding a drug-filled cavity (nanocapsules). Nanoparticles can penetrate into small capillaries, allowing enhanced accumulation of the encapsulated drug at target sites (Calvo et al., J Neurosci Methods 111(2):151-5 (2001)). Nanoparticles can passively target tumor tissue through enhanced permeation and retention effect (Monsky et al., Cancer Res. 59(16):4129-35 (1999); Stroh et al., Nat Med. 11(6):678-82 (2005)). Nanoparticles can be delivered to distant target sites either by localized catheter-based infusion (Panyam et al., J Drug Target 10(6):515-23 (2002)) or by attaching a ligand to nanoparticle surface that has affinity for a specific tissue (Shenoy et al., J Pharm Pharmacol 57(4):411-22 (2005)). Because of sustained release properties, nanoparticles can prolong the availability of the encapsulated drug at the target site, resulting in greater and sustained therapeutic effect.

In certain embodiments described herein, the nanoparticle formulations comprise novel excipients and/or novel active agents. In certain instances, engineering the nanoparticle to have a porous surface can increase the efficiency and/or efficacy of delivery of the active agent. In some embodiments the nanoparticle comprises the active agent and does not comprise an excipient. In certain embodiments, the active agent and/or the excipient can be approved by the U.S. Food and Drug Administration, as described in other sections herein.

Methods for Stabilizing Suspension-Based Formulations with Nanoparticles

Certain embodiments disclosed herein relate to compositions and methods related to stabilizing suspension-based formulations in HFA (for example, HFA227 or HFA134a), or other propellants with nanoparticles. In one particular embodiment, nanoparticles can be prepared that are capable of being well-dispersed in HFA or other propellants of pMDIs. In at least one embodiment, the nanoparticles can act to stabilize the suspension-based formulation by preventing flocculation of otherwise unstable colloidal drug particles.

HFA suspensions can be inherently unstable due to the aggregation tendency, the influence of gravity, the tendency to phase separation of particles, the attraction between drug particles, the attraction between drug particles and device surfaces, and other properties. Some methods for lending stability to HFA suspensions include, but are not limited to, density matching, modifying surface properties of the particles, engineering porous particles, and preparing nanosuspensions with relatively uniform size distribution of nanoparticles.

The nanoparticles described herein can be utilized in either water soluble (or hydrophilic) or water insoluble (or hydrophobic) formulations. In certain embodiments, the suspension-based formulation can contain an active ingredient, as described herein.

In certain embodiments, the nanoparticle can comprise the same or a similar chemical composition as the colloidal drug particles that are the active drug ingredients.

In certain embodiments disclosed herein, the nanoparticles are in the range of 1 nm, 5 nm, 10 nm, 15 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 350 nm, 400 nm, 450 nm, 500 nm, or any value therebetween or less than the approximate size.

In certain embodiments, the method for making nanoparticles related to the disclosure can include preparing chitosan nanoparticles by the nano-precipitation technique. In this respect, an oil-in-water method can be used as an alternative to double emulsion, in order to avoid the water-organic solvent interface during the first emulsification step. The surface of the nanoparticle can be modified, for example and without limitation by ring-opening polymerization of the lactide with the hydroxyl and amino groups on the chitosan nanoparticle surface as the initiator. In one embodiment, the nanoparticles have an average size of 100 nm.

In certain embodiments the dispersion stabilization can be stabilized by the compositions and methods disclosed herein. In certain embodiments, the sedimentation rate of micronized nanoparticles comprising drug ingredients can show increased stability. For example, micronized insulin in HFA227 utilizing the described chitosan-PLA nanoparticles improves the suspension formulation over the standard preparation methods and compositions. In certain embodiments, this stability can be a result of amphiphiles that impart stability to drug crystals by preferentially adsorbing at the drug-HFA interface.

### Porous Particles and Methods of Preparing the Same

Certain embodiments disclosed herein relate to novel methods for preparing or engineering porous drug particles, in one embodiment polar drug particles, which have long-term physical stability in HFA propellants, and pMDI formulations with enhanced aerosol characteristics.

The porous particles disclosed herein can include biocompatible and/or biodegradable polymers, copolymers, or blends thereof. In certain embodiments, the polymers include, without limitation, polylactides, polylactide-glycolides, cyclodextrins, polyacrylates, methylcellulose, carboxymethylcellulose, polyvinyl alcohols, polyanhydrides, polylactams, polyvinyl pyrrolidones, polysaccharides (dextran, starch, chitin, chitosan, etc.), hyaluronic acid, and/or proteins (albumin, collagen, gelatin, etc.). In certain embodiments the porous particles comprise nanoparticles as described herein.

In certain embodiments, the porous particle composition size includes a diameter of without limitation 0.5 micron, 1 micron, 5 microns, 10 microns, 15 microns, 20 microns, 30 microns, 40 microns, 50 microns, 60 microns, 70 microns, 80 microns, 90 microns, 100 microns, or any value therebetween or less than.

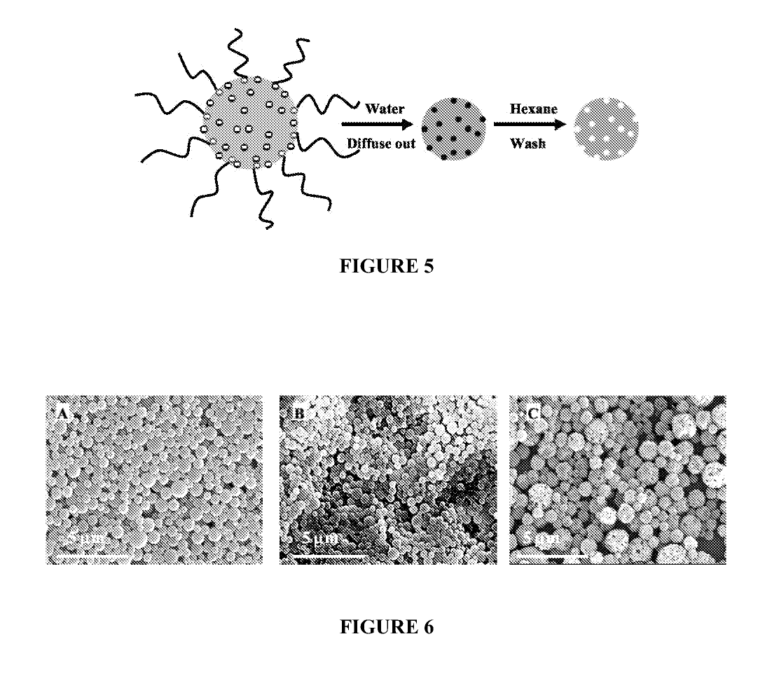

Traditional spray-drying techniques have been inadequate for preparing porous particles, and instead, the emulsification-diffusion process as depicted in FIG. 5 can provide superior results. In one particular embodiment, the particles can be formed by way of a modified emulsification-diffusion technique. In certain embodiments of the methods, the active agent can first be dissolved in an aqueous dispersion of lecithin or similar porosity agent or dispersion agent (which can be a solid, liquid, or gas), including without limitation, glycolipids, phospholipds, triglycerides, and other porosity agent such as poly(lactic acid), poly(∈-caprolactone), poly(lactic-co-glycolic acid), poly(ethylene glycol), poly(propylene glycol), cyclodextrin, (2-Hydroxyl propyl)-β-cyclodextrin and the like.

Next, the aqueous dispersion can be emulsified in a partially water-miscible organic solvent (such as without limitation ethyl acetate, methanol, ethanol, 1-propanol, 2-propanol, acetonitrile, N,N′-dimethylformamide, tetrahydrofuran, or the like) to obtain a water-in-oil (W/O) emulsion stabilized by a surfactant, such as, without limitation, sodium (bis-2-ethylhexyl) sulfosuccinate or aerosol-OT (AOT), or other surfactant or stabilizing compound. Other surfactants that can be used with certain embodiments include, but are not limited to, ionic or non-ionic surfactants such as phospholipids (naturally occurring or synthetic), glycolipids, ganglioside GM1, sphingomuelin, phosphatidic acid, cardiolipin, lipids bearing polymer changes such as polyethylene glycol, chitin, hyaluronic acid, polyvinylpyrrolidone, lipids bearing sulfonated mono-, di-, and polysaccharides, fatty acids such as palmitic acid, stearic acid, oleic acid, cholesterol, cholesterol esters, sorbitan esters, polyoxyethelene, oleyl polyoxyethylene ether, oleic acid, L81, glycerol esters, sucrose esters, lauryl polyoxyethylene ether, block copolymers, sodium sulfosuccinate, and the like.

Next, the emulsion can be diluted in a larger volume of the organic solvent. During this step, water can start to diffuse from within the droplets and into the organic phase. During the diffusion process, the negatively charged lecithin particles can become trapped inside the water droplets. Without wishing to be bound by theory, it is believed that the negatively charged lecithin particles become trapped inside water droplets due to the repulsive forces experienced with the head group of the surfactant (such as AOT) at the interface.

The trapped lecithin particles inside the drug particles provide a structure for the pores, and can then be removed later by washing (for example and without limitation, with hexane, or the like), thus resulting in a porous morphology with drug composition of more than without limitation 50 wt %, 55 wt %, 60 wt %, 65 wt %, 70 wt %, 75 wt %, 80 wt %, 85 wt %, 90 wt %, 91 wt %, 92 wt %, 93 wt %, 94 wt %, 95 wt %, 96 wt %, 97 wt %, 98 wt %, 99 wt %, or greater or any value therebetween by weight. The remaining composition is residual lecithin or other the like. In the case of lecithin, it is an FDA-approved excipient for drug formulations. Furthermore, characterization by NMR indicate that little to no AOT remains.

The physical stability and the aerosol characteristics of the porous particle formulations can be significantly improved compared to present commercially available formulations. Furthermore, the methods disclosed herein can provide an advantage over what is presently used in the art as the disclosed methods do not require the use of co-solvents in conjunction with one or more surfactant to attain similar (or inferior) dispersion stability. Co-solvents are not desirable since many potential problems can result with their use. Some potential problems with using co-solvents include: chemical instability of the drug substance, extraction of elastomeric components, as well as an unappealing taste for the recipient.

The methods disclosed herein also provide further advantages over what is known in the art, for example at U.S. Pat. No. 6,565,885; U.S. Patent Application Publication No. 2002037316; or WO/9966903, wherein the prepared porous particles result in an active drug ingredient content of less than 50 wt % or less by weight.

In addition, methods of the present disclosure typically yield compositions with bulk densities of less than about 0.5 g/cm³, 0.4 g/cm³, 0.3 g/cm³, 0.2 g/cm³, 0.1 g/cm³, 0.05 g/cm³, or any value therebetween. It is further appreciated that the particulate compositions disclosed herein can comprise structural matrices that can vary in morphology and structure and can have general pores, voids, hollows, or other indentations or perforations. For purposes described herein, the porosity of the particle microstructure can range from 0.1%, 0.2%, 0.3%, 0.4%, 0.5%, 1%, 2%, 3%, 4%, 5%, 10%, 20%, 30%, 40%, 50%, 60%, 70%, 80%, 90%, or greater or any value therebetween. Pore size can vary according to the specific goals of the particles, and is from 1 nm, 2 nm, 3 nm, 4 nm, 5 nm, 10 nm, 20 nm, 30 nm, 40 nm, 50 nm, 100 nm, 200 nm, 300 nm, 400 nm, or any value therebetween or less than.

In certain embodiments, the porous particle can comprise an active agent without an excipient. In certain embodiments the porous particles can comprise active agents and/or excipients that have been previously approved by the U.S. Food and Drug Administration, as described herein at other sections.

### Active Agents

In certain embodiments disclosed herein, the compounds prepared by the disclosed methods can contain at least one active ingredient or active agent. As used herein, “active agent” generally refers to an agent, drug, compound, composition of matter or mixture thereof which provides some diagnostic, prophylactic, therapeutic or pharmacologic, often beneficial effect. The active agents described herein can be utilized with the nanoparticle and/or the porous particle formulation. An active agent can include, without limitation, foods, food supplements, nutrients, drugs, vaccines, vitamins, and other beneficial agents or any physiologically or pharmacologically active substance that produces a localized or systemic effect in a subject.

Some other non-limiting examples of active agents include antibiotics, antibodies, antiviral agents, anepileptic agents, analgesics, anti-inflammatory agents and bronchodilators, and viruses and can be inorganic and organic compounds, including, without limitation, drugs which act on the peripheral nerves, adrenergic receptors, cholinergic receptors, the skeletal muscles, the cardiovascular system, smooth muscles, the blood circulatory system, synaptic sites, neuroeffector junction sites, endocrine and hormone systems, the immunological system, the reproductive system, the skeletal system, autacoid systems, the alimentary and excretory systems, the histamine system and the central nervous system. Suitable agents can be selected from, for example and without limitation, polysaccharides, steroids, hypnotics and sedatives, psychic energizers, tranquilizers, anticonvulsants, muscle relaxants, anti-Parkinson agents, analgesics, anti-inflammatory agents, muscle contractant agents, antimicrobial agents, anti-malarial agents, hormonal agents including contraceptives, sympathomimetics, amino acids, peptides, polypeptides, and proteins capable of eliciting physiological effects, diuretics, lipid regulating agents, anti-androgenic agents, anti-parasitic agents, neoplastic agents, anti-neoplastic agents, angiogenic agents, anti-angiogenic agents, hypoglycemic agents, nutritional agents and supplements, growth supplements, fats, anti-enteritis agents, electrolytes, vaccines and diagnostic agents.

Other examples of active agents useful in embodiments disclosed herein include, but are not limited to, at least one active ingredient, in one embodiment a polar drug ingredient. In some embodiments, the active drug ingredient can be soluble in the aqueous phase (for example, water and the excipients). In certain particular embodiments the active ingredient can be hydrophilic or hydrophobic. In some particular embodiments, an active drug ingredient can be one or more of, without limitation: salbutamol sulfate, terbutaline hemisulfate, therapeutic biomolecules, formoterol, corticosteroids, fluticasone, antibiotics (including tobramycin, ampicillin, amoxicillin, erythromycin, clarithromycin, azithromycin, tetracycline, fluoroquinolones, cefaclor, chlroamphenicol, ciproflaxicin, gentamicin, and others) steroids, chromolyn sodium, pain relievers (such as morphine, acetaminophen, ibuprofen, codeine, aspirin, ketoprofen, naproxen, etc.), vaccines (such as for tetanus, measles, mumps, rubella, hepatitis A, hepatitis B, flu, bubonic plague, cholera, smallpox, etc.) insulin, calcitonin, erythropoietin (EPO), Factor VIII, Factor, ceredase, cerezyme, cyclosporine, granulocyte colony stimulating factor (GCSF), alpha-1 proteinase inhibitor, elcatonin, granulocyte macrophage colony stimulating factor (GMCSF), growth hormone, human growth hormone (hGH), growth hormone releasing hormone (GHRH), heparin, low molecular weight heparin (LMWH), Interferon α, Interferon β, Interferon γ, Interleukin-2, luteinizing hormone releasing hormone (LHRH), leuprolide, somatostatin, somatostatin analogs including octreotide, vasopressin analog, follicle stimulating hormone (FSH), immunoglobulins, insulin-like growth factor, insulintropin, interleukin-1 receptor antagonist, interleukin-3, interleukin-4, interleukin-6, macrophage colony stimulating factor (M-CSF), nerve growth factor, parathyroid hormone (PTH), thymosin α-1, Interleukins (IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, etc.), interleukin receptors, respiratory syncytial virus antibody, cystic fibrosis transmembrane regulator (CFTR) gene, tetanus toxoid, lysozyme, other enzymes, deoxyribonuclease (Dnase), bactericidal/permeability increasing protein (BPI), anti-CMV antibody, 13-cis retinoic acid, nicotine, nicotine bitartrate, gentamicin, ciprofloxacin, amphotericin, amikacin, tobramycin, pentamidine isethionate, albuterol sulfate, metaproterenol sulfate, beclomethasone dipropionate, triamcinolone acetamide, budesonide acetonide, ipratropium bromide, flunisolide, fluticasone, fluticasone propionate, salmeterol xinofoate, formeterol fumarate, cromolyn sodium, ergotamine tartrate and the analogues, agonists and antagonists of the above.

Active agents can further comprise nucleic acids, present as without limitation bare nucleic acid molecules, viral vectors, associated viral particles, nucleic acids associated or incorporated within lipids or a lipid-containing material, plasmid DNA or RNA or other nucleic acid construction of a type suitable for transfection or transformation of cells, particularly cells of the alveolar regions of the lungs. In certain embodiments, the active agent can comprise a small molecular weight drug. In other embodiments, the active agent can comprise at least one large biomolecule, including but not limited to peptides, polypeptides, proteins, amino acids (including naturally occurring as well as non-natural amino acids or amino acid analogues), nucleotides, DNA, RNA, tRNA, mRNA, rRNA, shRNA, microRNA, and any combinations thereof, or the like. The active agents can be in various forms, including without limitation soluble and insoluble charged or uncharged molecules, components of molecular complexes or pharmacologically acceptable salts.

The active agents can be naturally occurring molecules or they can be recombinantly produced, or they can be analogs of the naturally occurring or recombinantly produced active agents with one or more amino acids added or deleted. Further, the active agent can comprise live attenuated or killed viruses suitable for use as vaccines.

### Excipients

In addition to the aforementioned materials and/or agents, other excipients can be added to a particulate composition to improve without limitation particle rigidity, production yield, emitted dose and/or deposition, shelf-life, subject compliance, or other objective. The excipients described herein can be utilized with the nanoparticle and/or the porous particle formulations described herein at other sections. In certain embodiments, the excipient can also comprise an active agent.

Some examples of optional excipients include, but are not limited to, coloring agents, taste-masking agents, buffers, hygroscopic agents, antioxidants, and chemical stabilizers. Some more specific examples include, but are not limited to, carbohydrates (monosaccharides, disaccharides, polysaccharides), such as dextrose (anhydrous and monohydrate), galactose, mannitol, D-mannose, sorbitol, sorbose, disaccharides (lactose, sucrose, maltose, trehalose, etc.), cyclodextrins, maltodextrins, and others. In addition, buffering agents or salts can also be included, such as, without limitation, inorganic (sodium chloride, etc.), organic acids and their salts (e.g. carboxylic acids, sodium citrate, sodium ascorbate, magnesium gluconate, sodium gluconate, tromethamine hydrochloride, ammonium carbonate, ammonium acetate, ammonium chloride, camphor, etc.).

While the dispersions disclosed herein are generally suitable for pulmonary administration, they can also be used locally or systemically for administration of compounds to any location of the body, and by any number of different routes including, but not limited to, the gastrointestinal tract, the respiratory tract, topical, intramuscular, intraperitoneal, nasal, vaginal, rectal, aural, oral, or ocular administration.

The particles described herein can be used for controlled systemic or local delivery of at least one active agent, as disclosed. Administration of the particle suspensions by pulmonary aerosolization, for example, can permit deep lung delivery of the active agent deep into the lung, and potentially into blood vessels therein.

### Treatment of Therapeutic Conditions and Diseases

Certain embodiments disclosed herein relate to compositions and methods relating to treating at least one therapeutic condition and/or disease with the compositions made by the disclosed methods. As used herein, “treat,” “treatment,” “treating,” and all derivations thereof can refer to preventing or ameliorating at least one symptom of a disease or condition in a subject, such as a mammal, and in one embodiment a human. In certain embodiments, at least one condition or disease can be related to a pulmonary condition or disease. In other particular embodiments, at least one condition or disease can be related to a systemic condition or disease. In other particular embodiments, at least one condition or disease can be related to a local condition or disease. In other particular embodiments, the compositions and/or methods described herein can relate to delivery of preventative drug formulations, including vaccines.

Further embodiments described herein relate to diagnosing a particular therapeutic condition and/or disease. Conventional methods can be employed for use with the particles disclosed herein for diagnostic purposes, for example and without limitation, positron emission tomography (PET), computer assisted tomography (CAT), single photon emission computerized tomography, x-ray, fluoroscopy, and magnetic resonance imaging (MRI).

In certain embodiments, the therapeutic condition and/or disease can be selected from one or more of, without limitation, chronic pulmonary diseases, lung cancer, cystic fibrosis, pulmonary fibrosis, asthma, bronchitis, pneumonia, pleurisy, emphysema, pulmonary fibrosis, diabetes, interstitial lung disease, sarcoidosis, chronic obstructive pulmonary disease, infant respiratory distress syndrome, adult respiratory distress syndrome, pulmonary actinomycosis, pulmonary alveolar proteinosis, pulmonary anthrax, pulmonary arteriovenous malformation, pulmonary edema, pulmonary embolus, pulmonary histiocytosis X (eosinophilic granuloma), pulmonary hypertension, pulmonary nocardiosis, pulmonary tuberculosis, pulmonary veno-occlusive disease, rheumatoid lung disease, hypertension, HIV-AIDS, leukemia, lymphoma, cancer, systemic vasculitis, anemia, stem cell transplants, hemophilia, polycythemia vera, thalassemia, thrombocytopenia, von Willebrand disease, arthritis, vascular diseases, heart conditions, heart disease, and others.

### EXAMPLES

### Example 1

### Preparation of Nanoparticles

Chitosan nanoparticles were prepared by standard nano-precipitation technique. Briefly, chitosan was dissolved in DI-water with the aid of hydrochloric acid at 80° C. Hydrogen peroxide (30%) was subsequently added into the chitosan aqueous solution to degrade the long chain chitosan to water-soluble smaller oligomers. The chitosan oligomer was then dissolved in water. The chitosan aqueous solution was added into a large volume of ethanol dropwise under mild stirring. Chitosan was then precipitated from the organic phase as nanoparticles because it is insoluble in ethanol.

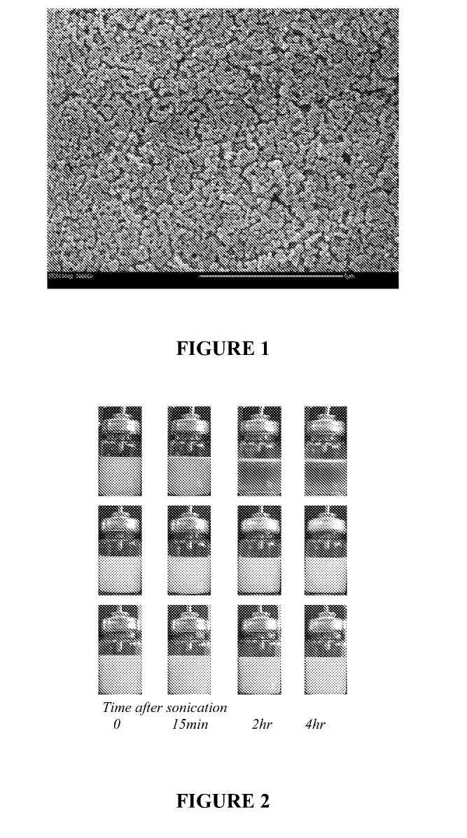

The surface of the chitosan nanoparticle was then modified by ring-opening polymerization of the lactide with the hydroxyl and amine groups on chitosan nanoparticle surface as the initiator. A scanning electron microscope image (SEM image) of the prepared nanoparticles is shown in FIG. 1. The nanoparticles have an average size of approximately 100 nm, although the size can controlled by varying the preparation parameters. parameters such as the concentration of chitosan aqueous solution, different type of organic solvents used and the presence of surfactant.

| Parameters | | Particle Size (nm) |
|---|---|---|
| Organic solvent | Ethanol | 100 |
| Chitosan concentration | Acetone | 150 |
| (25 mg/ml) | isopropyl alcohol | 120 |
| Chitosan concentration | 10 mg/ml | 85 |
| Organic solvent: Ethanol | 25 mg/ml | 100 |
| | 40 mg/ml | 120 |
| Surfactant used | None | 100 |
| Chitosan concentration (25 mg/ml) | PEO-PPO-PEO | 80 |
| Organic solvent: Ethanol | (Pluronic L64) | |

### Example 2

### Dispersion Stabilization

Sedimentation rate of micronized insulin in HFA 227 were conducted to test the physical stability of dispersions. As shown in FIG. 2, the pure insulin particles show poor stability in HFA227, creaming out in seconds and not being redispersible. Upon addition of chitosan-PLA nanoparticles, the stability of insulin is improved. In addition, the suspensions can be redispersed simply by hand-shaking even after several days of storage. The amphiphiles impart stability to drug crystals by preferentially adsorbing at the drug-HFA interface. Other active agents tested with similar results obtained included salbutamol sulfate (SS) and budesonide (BD).

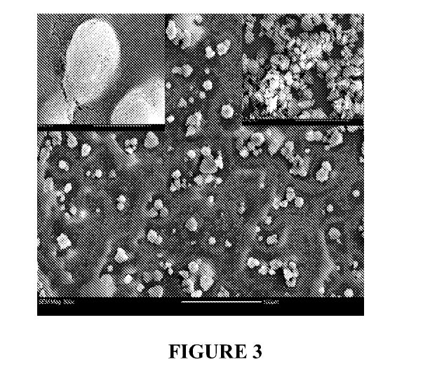

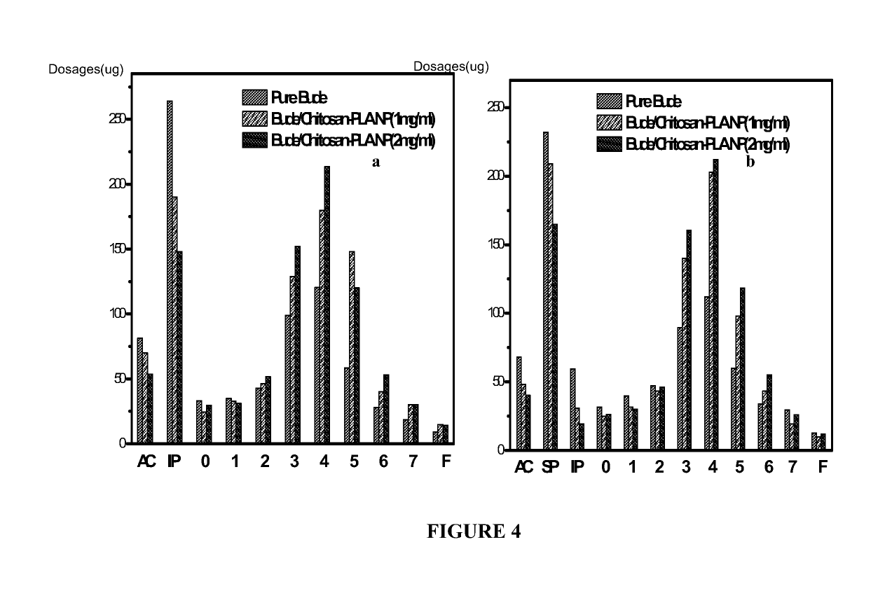

As can be seen in FIG. 3, insulin particles obtained after the physical stability studies in a model HFA propellant, HPFP indicate that the nanoparticles adsorbed onto the surface of large insulin particles. FIG. 4 indicates the aerosol characteristics of the formulations, including budesonide, and similar behavior was observed for salbutamol sulfate.

### Example 3

### Preparation of Porous Particles

HFA134a and HFA227 (>99.99% purity), and 2H, 3H-perfluoropentane (HPFP) (>98% purity) were used for preparation of the salbutamol sulfate (SS) particles by emulsification-diffusion. Briefly, 25 mg of SS was dissolved in 0.8 ml water. The aqueous solution was emulsified in 19 ml ethyl acetate using a sonication bath at 303K. A water-in-oil (W/O) emulsion was obtained, and was subsequently added to a large volume (150 ml) of ethyl acetate. SS particles were formed as water that makes the dispersed emulsion phase diffuses out into ethyl acetate and collected by centrifugation.

Porous SS particles were also prepared by emulsification diffusion. Briefly, 25 mg of SS was dissolved in 0.8 ml lecithin aqueous dispersion. The lecithin particles used here have an effective particle diameter of 270 nm and polydispersity of 0.295 with zeta potential of −43.4 my. The SS solution was then emulsified with 19 ml of AOT solution in ethyl acetate of various concentrations at 303K. The obtained W/Ac emulsion was then transferred into 150 ml ethyl acetate, and the precipitated SS particles were collected by centrifugation, washed with hexane twice to remove any residue lecithin and AOT, then dried at room temperature to give porous SS particles.

### Example 4

### Assessing Particle Properties

### Interfacial Tension

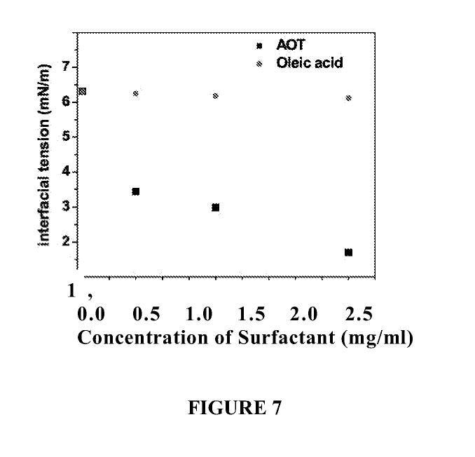

The interfacial tension (γ) between water (saturated with ethyl acetate) and ethyl acetate (saturated with water) with or without presence of surfactants was measured using a pendant drop tensiometer. Measurements were carried out inside a sealed cuvette at 298 K. Because no experimental density values of the mutually saturated phases are available in the literature, the density of pure water and ethyl acetate was used to calculate the ã. As indicated in FIG. 7, the interfacial tension of water/ethyl acetate remained almost unchanged with the introduction of oleic acid, while the presence of AOT can reduce the tension to a significant extent. The tension measurements indicate that oleic acid does not reside at the interface of ethyl acetate and water in the emulsion, which keeps it from generating the desired porous morphology.

### Example 5

### Assessing Particle Properties

### Particle Size and Morphology

The SS particles size and morphology were analyzed by scanning electron microscopy (SEM, Hitachi S-2400). Several drops of the particle suspension in HPFP were placed on a cover glass slip and allowed to dry. The cover glass substrates were then sputtered for 30 s with gold for SEM analysis. The SS content in the porous particles was quantified using UV spectroscopy with 0.1 M NaOH methanol solution as the solvent and detection wavelength of 246 nm. As indicated in FIG. 6, AOT gives rise to the porous morphology for SS particles. The porosity of the particles is in direct proportion to the AOT concentration used. Thus, it is expected that an increased amount of AOT surfactant molecules at the interface of the ethyl acetate would produce stronger repulsive forces with the lecithin particles inside the water droplets, which results in more lecithin particles trapped, and enhancing the porosity of the particles.

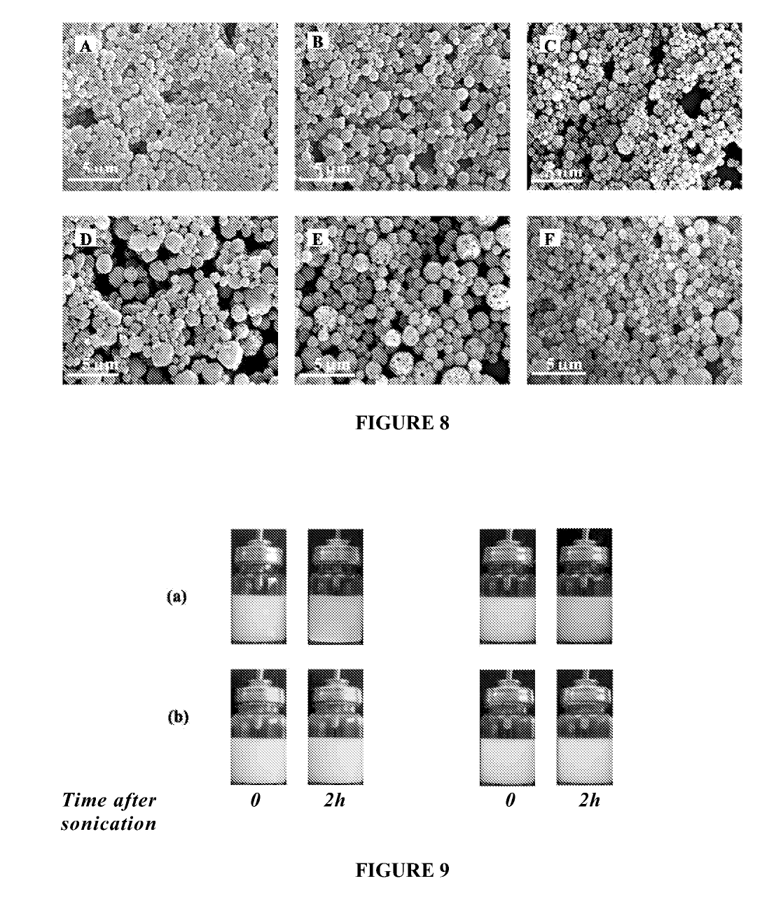

Likewise the effect of lecithin particle concentration on the particle morphology is shown in FIG. 8. At two different fixed AOT concentrations, the greater the concentration of lecithin, the greater the porosity obtained.

The compositions of porous SS particles were analyzed by UV spectroscopy. The content of SS in the porous particles varied from 78 to 91 wt % with the concentration of AOT and lecithin used in the preparation process. In addition, terbutaline hemisulfate porous particles were produced in the same method, with similar results.

### Example 6

### Assessing Particle Properties

Sedimentation rate experiments of the porous SS particles were performed in HFA134a and HFA227 at 298 K and saturation pressure of the propellant. Colloidal stability of solid SS particles prepared without AOT and lecithin was also tested as a comparison. As shown in FIG. 9, solid SS spheres obtained from emulsification-diffusion had poor stability in the hydrofluoroalkane propellant. Creaming of the particles in HFA227 or sedimentation in HFA134a started taking place immediately after mechanical input used for dispersing the particles stopped. Stability is achieved with the porous SS particles in both HFA227 and HFA134a shown in the figure, as a result of the penetration of propellant into the porous particles which give rise to both a close density of the particle with the propellant and reduced van der Waal attractive forces between the particles.

Further, an exact mass of the drug particles were initially fed into pressure proof glass vials and crimp-sealed with 50 μl metering valves (EPDM Spraymiser™, 3M Inc). Subsequently, a known amount of HFA227 or HFA134a was added with the help of a manual syringe pump (HiP 50-6-15) and a high pressure aerosol filler, to make a 2 mg/ml drug concentration in the propellant HFAs. The dispersions were then sonicated in a low energy sonication bath for 30 min in order to break up any aggregates. The physical stability of the suspensions in HFAs was investigated by visually monitoring the dispersion as a function of time after mechanical energy input ceased.

### Example 7

### Assessing Particle Properties

### Aerosol Performance

The aerosol properties of the solid SS and porous SS core-shell formulations were determined with an Andersen Cascade Impactor (ACI, CroPharm, Inc.) operated at a flow rate of 28.3 L/min. The experiments were carried out at 298 K and 45% relative humidity. Before each test, several shots were first fired to waste, then 10 shots were released into the impactor, with an interval of 30 s between actuations. Three independent canisters were tested for each formulation. The average and standard deviation from those three independent runs were tabulated. The amount of drug deposited on the valve stem, actuator, induction port and stages was rinsed thoroughly with a known volume of 0.1 M NaOH methanol solution. The drug content was then quantified by UV spectroscopy, with a detection wavelength of 246 nm. The effect of a spacer on the aerosol characteristics was investigated. The results obtained with the formulations proposed here are contrasted with those obtained with Ventolin® HFA (GlaxoSmithKline). The same actuator as that of Ventolin® HFA was used in all experiments. The fine particle fraction (FPF) is defined as the percentage of drug on the respirable stages of the impactor (stage 3 to terminal filter) over the total amount of drug released into the device (from the induction port to filter). Fine particle dose (FPD) is the mass of drug on the respirable stages.

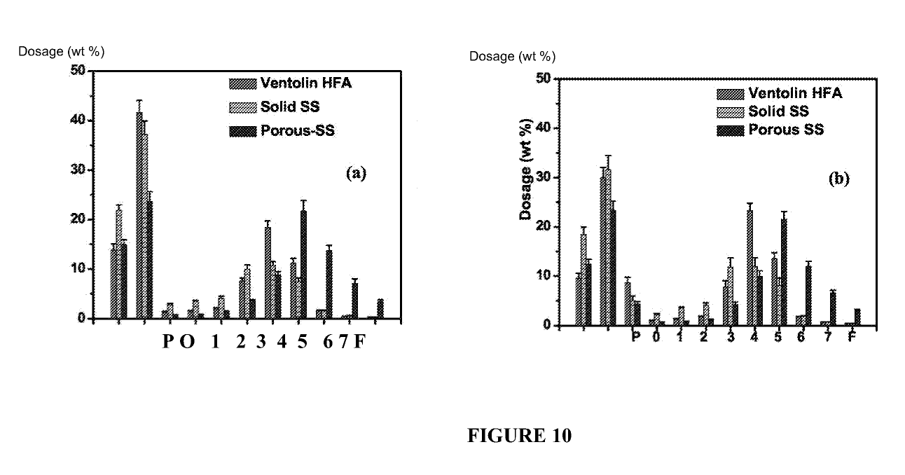

The results are shown in FIG. 10 and Table I. The amount retained at each stage of the ACI is reported as dosage percentage of the total amount of drug delivered from the pMDI. As indicated in FIG. 10 and Table I, the aerosol performance of the porous SS formulation is significantly improved compared to both the commercial formulation and the bare SS particles formed using the emulsification-diffusion technique described herein. The particle FGF_{4.7 μm} for the porous SS formulation in the presence of a spacer is further improved.

TABLE 1
Aerodynamic properties of various SS formulations in HFA134a
as probed by the ACI test (10 × actuation dose)

| | Ventolin HFA | | Solid-SS | | Porous SS | |
|---|---|---|---|---|---|---|
| | (n = 3) | | (n = 3) | | (n = 3) | |
| | No | With | No | With | No | With |
| | Spacer | spacer | spacer | spacer | spacer | spacer |
| FPF(<5.8 μm) % | 48.3 + 3.5 | 81.8 + 4.2 | 44.3 + 3.0 | 78.0 + 3.5 | 70.3 + 3.1 | 91.0 + 1.5 |
| FPF(<4.7 μm) % | 45.9 + 2.4 | 78.7 + 2.9 | 39.1 + 3.1 | 69.7 + 3.0 | 68.6 + 2.1 | 89.0 + 2.4 |
| FPF(<3.3 μm) % | 37.1 + 3.0 | 65.7 + 2.0 | 26.3 + 2.5 | 46.1 + 1.9 | 64.5 + 1.9 | 82.4 + 2.0 |
| FPD(<4.7 μm) μg | 46.7 + 4.5 | 57.6 + 4.0 | 26.8 + 3.4 | 31.1 + 3.5 | 53.0 + 3.5 | 51.5 + 2.5 |
| FPD(<3.3 μm) μg | 37.8 + 3.3 | 48.1 + 3.1 | 18.1 + 2.8 | 20.6 + 2.6 | 49.8 + 2.6 | 47.7 + 2.4 |
| MMAD(μm) | 2.5 ± 0.1 | 2.3 ± 0.1 | 3.0 ± 0.2 | 2.8 ± 0.2 | 1.4 + 0.1 | 1.4 + 0.1 |
| GSD (μm) | 1.9 ± 0.1 | 1.8 ± 0.1 | 2.1 ± 0.1 | 2.1 ± 0.2 | 2.1 + 0.2 | 2.1 + 0.1 |

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods disclosed herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are disclosed herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically disclosed herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A process for preparing an active agent porous particle, the process comprising contacting at least one active agent with an aqueous dispersion, emulsifying the active agent and aqueous dispersion, and diffusing the active agent and aqueous dispersion in an organic solvent under suitable conditions to form an active agent porous particle.

2. The process of claim 1 wherein the suitable conditions are one or more of: the presence of at least one surfactant, and the presence of at least one porosity agent.

3. The process of claim 2 wherein the porosity agent is one or more of: lecithin, glycolipids, phospholipids, and triglycerides.

4. The process of claim 2 wherein the surfactant is one or more of ionic or non-ionic surfactants: phospholipids, glycolipids, ganglioside GM1, sphingomuelin, phosphatidic acid, cardiolipin, lipids bearing polymer changes such as polyethylene glycol, chitin, hyaluronic acid, polyvinylpyrrolidone, lipids bearing sulfonated mono-, di-, and polysaccharides, fatty acids such as palmitic acid, stearic acid, oleic acid, cholesterol, cholesterol esters, sorbitan esters, polyoxyethelene, oleyl polyoxyethylene ether, glycerol esters, sucrose esters, lauryl polyoxyethylene ether, block copolymers, sodium (bis-2-ethylhexyl) sulfosuccinate AOT.

5. The process of claim 1 wherein the active agent is one or more of: antibiotics, antibodies, antiviral agents, anepileptic agents, analgesics, anti-inflammatory agents and bronchodilators, polysaccharides, steroids, hypnotics and sedatives, psychic energizers, tranquilizers, anticonvulsants, muscle relaxants, anti-Parkinson agents, analgesics, anti-inflammatory agents, muscle contractant agents, antimicrobial agents, anti-malarial agents, hormonal agents including contraceptives, sympathomimetics, amino acids, peptides, polypeptides, and proteins capable of eliciting physiological effects, diuretics, lipid regulating agents, anti-androgenic agents, anti-parasitic agents, neoplastic agents, anti-neoplastic agents, angiogenic agents, anti-angiogenic agents, hypoglycemic agents, nutritional agents and supplements, growth supplements, fats, anti-enteritis agents, electrolytes, vaccines, salbutamol sulfate, terbutaline hemisulfate, therapeutic biomolecules, formoterol, corticosteroids, fluticasone, chromolyn sodium, pain relievers insulin, calcitonin, erythropoietin, Factor VIII, Factor, ceredase, cerezyme, cyclosporine, granulocyte colony stimulating factor, alpha-1 proteinase inhibitor, elcatonin, granulocyte macrophage colony stimulating factor, growth hormone, human growth hormone, growth hormone releasing hormone, heparin, low molecular weight heparin, Interferon α, Interferon β, Interferon γ, Interleukin-2, luteinizing hormone releasing hormone, leuprolide, somatostatin, somatostatin analogs including octreotide, vasopressin analog, follicle stimulating hormone, immunoglobulins, insulin-like growth factor, insulintropin, interleukin-1 receptor antagonist, interleukin-3, interleukin-4, interleukin-6, macrophage colony stimulating factor, nerve growth factor, parathyroid hormone, thymosin α-1, Interleukins, interleukin receptors, soluble cytokines, soluble cytokine receptors, respiratory syncytial virus antibody, tetanus toxoid, lysozyme, other enzymes, deoxyribonuclease, bactericidal/permeability increasing protein, anti-CMV antibody, 13-cis retinoic acid, nicotine, nicotine bitartrate, gentamicin, ciprofloxacin, amphotericin, amikacin, tobramycin, pentamidine isethionate, albuterol sulfate, metaproterenol sulfate, beclomethasone dipropionate, triamcinolone acetamide, budesonide acetonide, ipratropium bromide, flunisolide, fluticasone, fluticasone propionate, salmeterol xinofoate, formeterol fumarate, cromolyn sodium, ergotamine tartrate, nucleic acids, peptides, polypeptides, proteins, amino acids nucleotides, DNA, RNA, tRNA, mRNA, rRNA, shRNA, microRNA, and pharmacologically acceptable salts thereof.

6. The process of claim 1 wherein the organic solvent is one or more of: ethyl acetate, methanol, ethanol, 1-propanol, 2-propanol, acetonitrile, N,N′-dimethylformamide, tetrahydrofuran.

7. (canceled)

8. A process for preparing an active agent porous particle, the process comprising contacting an active agent with an aqueous dispersion, emulsifying the active agent and aqueous dispersion in the presence of AOT, and diffusing the active agent and aqueous dispersion in an organic solvent comprising ethyl acetate under suitable conditions to form an active agent porous particle, wherein the active agent comprises a therapeutic drug.

9-10. (canceled)

11. A process for preparing a stabilized suspension-based aerosolized formulation, the process comprising preparing nanoparticles, modifying the surface of the nanoparticles, and suspending the nanoparticles in an aerosolized formulation comprising at least one active agent, thereby preparing a stabilized suspension-based aerosolized formulation.

12. The process of claim 11 wherein the diameter of the nanoparticles comprise about 1 nm, 5 nm, 10 nm, 15 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 350 nm, 400 nm, 450 nm, or 500 nm in size.

13. The process of claim 11 wherein the aerosolized formulation further comprises hydrofluoroalkane propellant.

14. The process of claim 11 wherein the at least one active agent is one or more of: insulin, budesonide, salbutamol sulfate, and terebutaline hemisulfate.

15. (canceled)
